(19) United States
(12) Patent Application Publication
(10) Pub. No.: US 2019/0378600 A1
(43) Pub. Date: Dec. 12, 2019

(54) MEDICINE MANAGEMENT APPARATUS AND MEDICINE MANAGEMENT METHOD

(71) Applicant: HITACHI, LTD., Tokyo (JP)

(72) Inventors: Takaaki KIMURA, Tokyo (JP); Kota OTAKE, Tokyo (JP)

(21) Appl. No.: 16/424,911

(22) Filed: May 29, 2019

(30) Foreign Application Priority Data
Jun. 12, 2018 (JP) 2018-111841

(51) Int. Cl. G16H 20/10; G06Q 10/08; G16H 40/20; G06Q 10/06

(52) U.S. Cl. CPC G16H 20/10; G06Q 10/0875; G06Q 30/018; G06Q 10/06; G16H 40/20

(57) ABSTRACT

A medicine management apparatus has a production management apparatus that manages lot information including a lot number of a medicine and lot trace information; a change management apparatus that manages change control information including a change control number of the medicine and product information; and an input apparatus that inputs the lot number of the medicine and the change control number, the medicine management apparatus including: a first storage unit that stores the lot information r; a second storage unit that stores the lot trace information; a third storage unit that stores the change control information; and a data management unit that manages the lot information including the lot number and the lot trace information in association with the change control information including the change control number according to the lot number and the change control number to be applied to a product having the lot number that is input.

### CROSS-REFERENCE TO RELATED APPLICATION

The present application claims priority from Japanese application JP 2018-111841, filed on Jun. 12, 2018, the contents of which is hereby incorporated by reference into this application.

### BACKGROUND OF THE INVENTION

### 1. Field of the Invention

The present invention relates to a medicine management apparatus and a medicine management method.

### 2. Description of the Related Art

In many pharmaceutical manufacturing companies, currently, one product is often sold to a plurality of countries along with the globalization of business. In many countries, there are strict regulations by the law on medicine manufacturing methods, and thus, it is necessary to obtain approval for a manufacturing method for each country. As a result, it is obliged to manufacture a medicine by the manufacturing method approved by an importing country. Thus, it is necessary to prepare a plurality of versions having different manufacturing method even for one medicine.

Meanwhile, pharmaceutical manufacturing companies usually manufacture a medicine at a plurality of manufacturing plants. Therefore, it is necessary to strictly manage the medicine manufactured at each plant in order to export the medicine manufactured by an appropriate manufacturing method to an appropriate country.

As a method for managing manufacturing of a drug related to such a situation, for example, a device (see JP 2008-525895 A) that enables registration of dimensions of a plurality of data in a manufacturing method is presented.

### SUMMARY OF THE INVENTION

Conventionally, a simple manufacturing record is used when exporting a medicine, and thus, it is difficult to confirm whether a manufacturing method that has been used for the product to be exported is a method that has been approved in an importing country. Thus, there occurs an accident that a medicine that has been manufactured by a manufacturing method unapproved in a country is exported to the country.

Therefore, an object of the invention is to provide a medicine management apparatus and a medicine management method that prevent a medicine that has been manufactured by a manufacturing method unapproved in a country from being exported to the country.

One embodiment of the invention is a medicine management apparatus connected so as to be capable of receiving information from: a production management apparatus that manages lot information including a lot number of a medicine and lot trace information; a change management apparatus that manages change control information including a change control number of the medicine, an importing country, and product information; and an input apparatus that inputs the lot number of the medicine and the change control number. The medicine management apparatus includes: a first storage unit that stores the lot information received from the production management apparatus; a second storage unit that stores the lot trace information received from the production management apparatus; a third storage unit that stores the change control information received from the change management apparatus; and a data management unit that manages the lot information including the lot number and the lot trace information in association with the change control information including the change control number according to the lot number and the change control number to be applied to a product having the lot number input from the input apparatus. The medicine management apparatus may further include a fourth storage unit that stores the lot number and the change control number to be applied to the product having the lot number input from the input apparatus. Further, first lot information out of the lot information stored in the first storage unit may be updated by second lot information out of the lot information received from the production management apparatus, and the data management unit may manage the second lot information in association with the change control information associated with the first lot information. Further, in the medicine management apparatus, first lot trace information out of the lot trace information stored in the second storage unit may be updated by second lot trace information out of the lot trace information received from the production management apparatus, and the data management unit may manage the second lot trace information in association with the change control information associated with the first lot trace information. Further, in the medicine management apparatus, first change control information out of the change control information stored in the third storage unit may be updated by second change control information out of the change control information received from the change management apparatus, and the data management unit may manage the second change control information in association with the lot information and the lot trace information associated with the first change control information. Further, in the medicine management apparatus, the data management unit may manage the change control information including the change control number to be applied to a predetermined product in association with the lot information of a raw material of the product and/or a processed product. Further, in the medicine management apparatus, the lot information includes item information, and the data management unit may manage the change control information including the change control number to be applied to a predetermined product in association with the lot information of a product including the same item information as the product. Further, in the medicine management apparatus, the lot trace information includes movement information, and the data management unit may manage the change control information including the change control number to be applied to a predetermined product in association with the lot information of the product before or after movement. Further, in the medicine management apparatus, the association of the change control information with the lot information of the product before or after the movement may be released when the movement information is deleted. Further, an output apparatus may be connected to the medicine management apparatus so as to be capable of receiving information.

Another embodiment of the invention is a medicine management method using a medicine management apparatus connected so as to be capable of receiving information from: a production management apparatus that manages lot information including item information and a lot number of a medicine and lot trace information; a change management apparatus that manages change control information including a change control number of the medicine, an importing country, and product information; and an input apparatus that inputs the lot number of the medicine and the change control number. The medicine management method includes: receiving the lot information from the production management apparatus; receiving the lot trace information from the production management apparatus; receiving the change control information from the change management apparatus; and associating the lot information including the lot number and the lot trace information with the change control information including the change control number according to the lot number and the change control number to be applied to a product having the lot number input from the input apparatus.

A still another embodiment of the invention is a program that causes a computer, connected so as to be capable of receiving information from a production management apparatus that manages lot information including item information and a lot number of a medicine and lot trace information, a change management apparatus that manages change control information including a change control number of the medicine, an importing country, and product information, and an input apparatus that inputs the lot number of the medicine and the change control number, to execute: receiving the lot information from the production management apparatus; receiving the lot trace information from the production management apparatus; receiving the change control information from the change management apparatus; and associating the lot information including the lot number and the lot trace information with the change control information including the change control number according to the lot number and the change control number to be applied to a product having the lot number input from the input apparatus. A storage medium storing the program to be readable manner by a computer is also an embodiment of the invention.

According to the invention, it is possible to provide the medicine management apparatus and the medicine management method that prevent a medicine that has been manufactured by a manufacturing method unapproved in a country from being exported to the country.

### BRIEF DESCRIPTION OF THE DRAWINGS

FIG. 1 is a schematic diagram illustrating an overall configuration of a medicine management apparatus according to an embodiment of the invention, a change information management apparatus, a production information management apparatus, and an input apparatus to which the medicine management apparatus is connected such that reception is possible, and an output apparatus to which the medicine management apparatus is connected such that transmission is possible;

FIG. 2 is a schematic diagram illustrating an example of an internal configuration of a medicine management apparatus according to an embodiment of the invention; and

FIG. 3 is a schematic view illustrating items constituting change control information received from the change management apparatus, items constituting lot information received from the production management apparatus, items constituting lot trace information received from the production management apparatus, items constituting change application information obtained from the input apparatus, and items constituting change control number information which has been already applied to each manufacturing lot number.

### DESCRIPTION OF THE PREFERRED EMBODIMENTS

An object, a feature, an advantage, and an idea of the invention are apparent to the person skilled in the art with the description in the present specification, and the person skilled in the art can easily reproduce the invention based on the description of the present specification. Embodiments and specific examples of the invention described below illustrate preferred embodiments of the invention, and are given for the illustration or description, and there is no intention to limit the invention thereto. It is apparent to those skilled in the art that various modifications and alterations can be made based on the description of the present specification within an intention and a scope of the invention disclosed in the present specification.

==Medicine Management Apparatus==

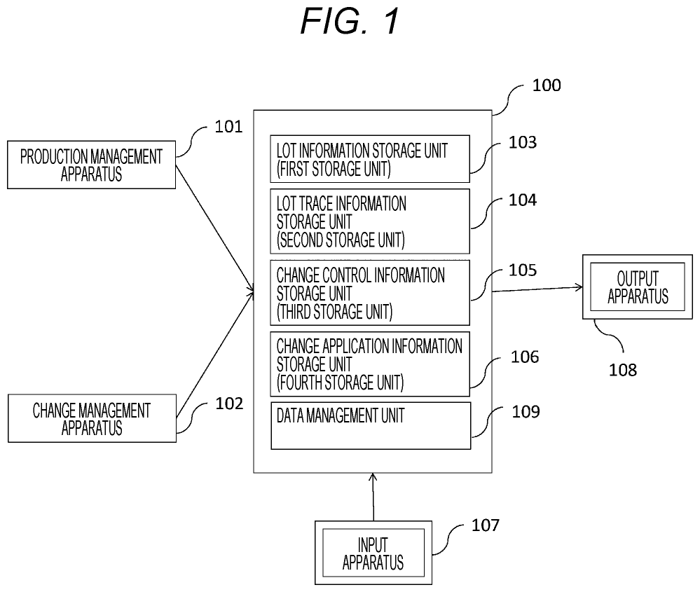

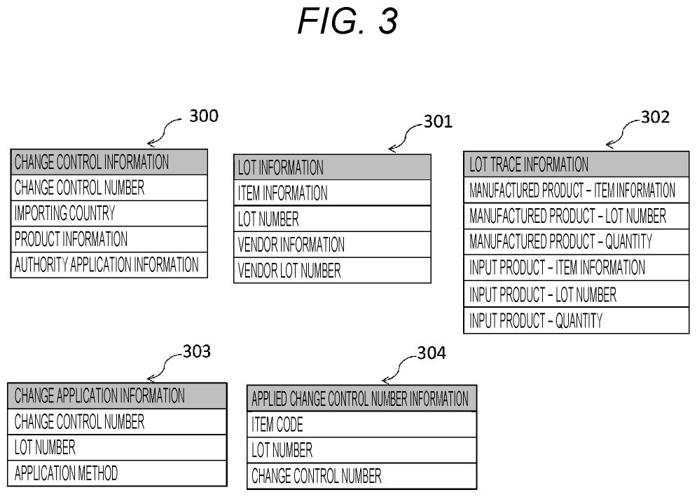

Hereinafter, a configuration of a medicine management apparatus 100 according to an embodiment of the invention will be described using FIGS. 1 to 3. Incidentally, the apparatus 100 may be configured using a so-called computer.

First, as illustrated in FIG. 1, the medicine management apparatus 100 according to the embodiment of the invention is connected so as to be capable of receiving from a production management apparatus 101 that manages lot information 301 including a lot number of a medicine and lot trace information 302, a change management apparatus 102 that manages change control information 300, and an input apparatus 107 that inputs the lot number of the medicine and a change control number, and includes: a first storage unit 103 that stores the lot information received from the production management apparatus; a second storage unit 104 that stores the lot trace information received from the production management apparatus; a third storage unit 105 that stores the change control information received from the change management apparatus; and a data management unit 109 that manages the lot information 301 including the lot number and the lot trace information 302 in association with the change control information 300 including the change control number according to change application information 303 including the lot number and the change control number to be applied to a product having the lot number input from the input apparatus.

The lot information 301 is information including the lot number of the medicine, and may include item information, vendor information, a vendor lot number, and the like. The item information is information (such as a product name, a medicine name, a compound name, or the like) that identifies the medicine, and may be managed by an item code. The vendor information is information including a company name, an address, and the like of a vendor of a raw material, and may be managed by a vendor code. The vendor lot number is a lot number used by a vendor company. Since the vendor information is added, it is possible to take over manufacturing information from a previous plant when one medicine is manufactured across a plurality of plants.

The lot trace information 302 includes information on a manufacturing process and a moving process of a lot. For example, the lot trace information 302 include item information, lot numbers, quantities, and vendor information on an input product input in a series of manufacturing processes to manufacture a product and a manufactured product manufactured using the product, movement information of a medicine, and the like.

The production management apparatus 101 is an apparatus that manages information on manufacturing of a medicine and movement of the medicine, and is installed, for example, in a product management department of a pharmaceutical company. When a raw material of a target product is purchased, data included in the lot information 301 or the lot trace information 302 is manually or automatically input to the production management apparatus 101.

The change control information 300 includes information such as a change control number, item information, an importing country, and pharmaceutical application information in the country. A change herein means a change in a manufacturing method of a medicine described in item information. The pharmaceutical application information includes information on what kind of manufacturing method has been applied in the country, whether the method has been approved or not, and the like.

The change management apparatus 102 is an apparatus that manages information related to the change in manufacturing method, such as the change control information 300, and is installed in, for example, a pharmaceutical application department of a pharmaceutical company. Data such as the change control information 300 is manually or automatically input to the change management apparatus 102 when pharmaceutical application information in another country is obtained.

The input apparatus 107 is an apparatus that inputs the change application information 303 including a lot number of a medicine, a change control number, and an application method at the time of manufacturing the target medicine, and is installed in a manufacturing plant where the medicine is actually manufactured.

The medicine management apparatus 100 is connected to be capable of receiving information from the production management apparatus 101, the change management apparatus 102, and the input apparatus 107. This connection may be performed so as to enable bidirectional transmission and reception of information.

The medicine management apparatus 100 may include a lot information reception unit 221, a lot trace information reception unit 225, a change control information reception unit 211, a change application information reception unit 271, a first storage unit 103, a second storage unit 104, a third storage unit 105, a fourth storage unit 106, and a data management unit 109. Although each reception unit may be independent, one reception device may also serve as a plurality of reception units. Although each storage unit may be also independent, one storage device may also serve as a plurality of storage units.

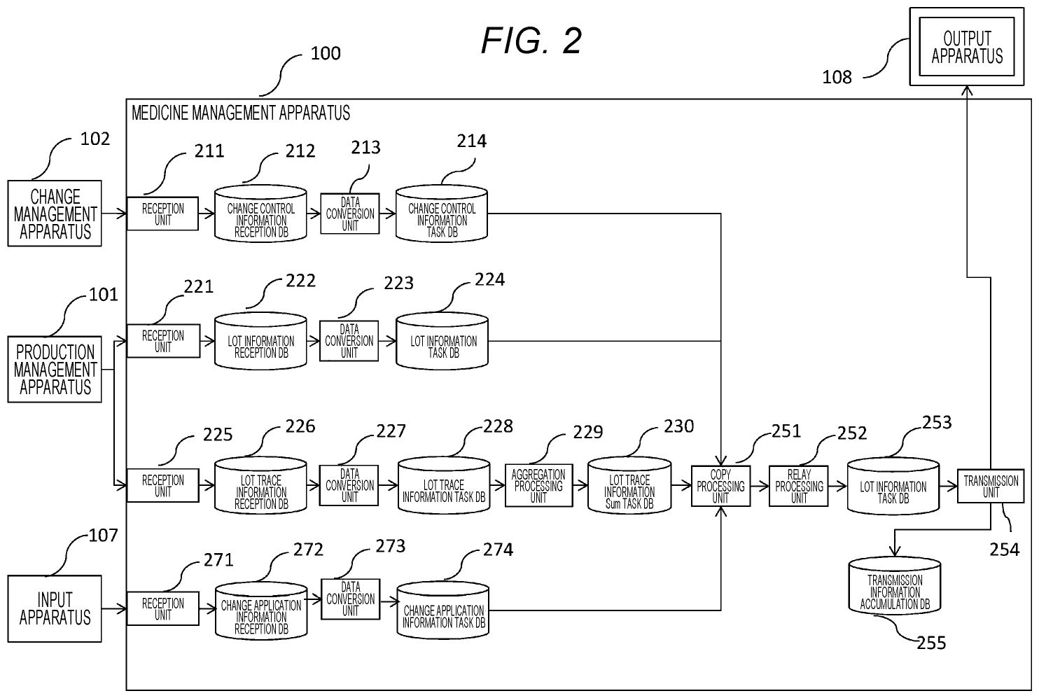

The medicine management apparatus 100 receives the lot information 301 from the production management apparatus 101 using the lot information reception unit 221, and stores the received lot information 301 in the first storage unit 103. Although a configuration of the first storage unit 103 is not particularly limited as long as the received lot information 301 can be stored and managed, and the first storage unit 103 may be constituted by a lot information reception database 222, a data conversion unit 223, and a lot information task database 224, for example, as illustrated in FIG. 2. In this case, the lot information 301 received by the lot information reception unit 221 is temporarily stored in the lot information reception database 222. If this data is formed of a plurality of code systems or character types, the data conversion unit 223 unifies the code systems or character types and stores the converted data in the lot information task database 224.

Further, the medicine management apparatus 100 receives the lot trace information 302 from the production management apparatus 101 using the lot trace information reception unit 225, and stores the received lot trace information 302 in the second storage unit 104. Although a configuration of the second storage unit 104 is not particularly limited as long as the received lot trace information 302 can be stored and managed, the second storage unit 104 may be constituted by a lot trace information reception database 226, a data conversion unit 227, a lot trace information task database 228, an aggregation processing unit 229, and a lot trace information sum task database 230, for example, as illustrated in FIG. 2. In this case, the lot trace information 302 received by the lot trace information reception unit 225 is temporarily stored in the lot trace information reception database 226. If this data is formed of a plurality of code systems or character types, the data conversion unit 227 unifies the code systems or character types and stores the converted data in the lot trace information task database 228. The lot trace information 302 includes initial registration, a decrease, an increase, cancellation, and the like, and thus, the aggregation processing unit 229 may aggregate related processes and calculate how many products have actually been moved or the like, and a calculated result may be added to the lot trace information 302 in the lot trace information task database 228 to be stored and managed by the lot trace information sum task database 230.

Further, the medicine management apparatus 100 receives the change control information 300 from the change management apparatus 102 using the change control information reception unit 211, and stores the received change control information 300 in the third storage unit 105. Although a configuration of the third storage unit 105 is not particularly limited as long as the received change control information 300 can be stored and managed, the third storage unit 105 may be constituted by a change control information reception database 212, a data conversion unit 213, and a change control information task database 214, for example, as illustrated in FIG. 2. In this case, the change control information 300 received by the change control information reception unit 211 is temporarily stored in the change control information reception database 212. If this data is formed of a plurality of code systems or character types, the data conversion unit 213 unifies the code systems or character types and stores the converted data in the change control information task database 214.

Further, the medicine management apparatus 100 receives the change application information 303 from the input apparatus 107 using the change application information reception unit 271, and stores the received change application information 303 in the fourth storage unit 106. Although a configuration of the fourth storage unit 106 is not particularly limited as long as the received change application information 303 can be stored and managed, the fourth storage unit 106 may be constituted by a change application information reception database 272, a data conversion unit 273, and a change application information task database 274, for example, as illustrated in FIG. 2. In this case, the change application information 303 received by the change application information reception unit 271 is temporarily stored in the change application information reception database 272. If this data is formed of a plurality of code systems or character types, the data conversion unit 273 unifies the code systems or character types and stores the converted data in the change application information task database 274.

The medicine management apparatus 100 further includes the data management unit 109. The data management unit 109 manages the lot information 301 including a lot number and the lot trace information 302 in association with the change control information 300 including a change control number according to the change application information 303 including the lot number input from the input apparatus, the change control number to be applied to a product having the lot number, an application method, and the like. Although a configuration of the data management unit 109 is not particularly limited as long as the lot information 301 and the lot trace information 302 and change control information 300 can managed in association with each other according to the change application information 303, the data management unit 109 may be constituted by a copy processing unit 251, a relay processing unit 252, and a lot information task database 253, for example, as illustrated in FIG. 2. The copy processing unit 251 applies the change control number to the lot information 301 and the lot trace information 302 based on the change application information 303 to be associated with the change control information 300. Further, the copy processing unit 251 may associate the change control information 300 with lot information of a product including the same item information as the lot information 301 and the lot trace information 302. Accordingly, the change control information 300 is associated with each lot in the same item. In the relay processing unit 252, for example, the change control information 300 may be associated with lot information of a raw material and/or a processed product included in the lot trace information 302. As a result, the change control information 300 is associated with related products such as a material, the raw material, and the processed product in a manufacturing process. Alternatively, the change control information 300 may be associated with lot information of a product before or after movement regarding movement information included in the lot trace information 302 in the relay processing unit 252. As a result, even if the related product or the same product is moved, the change control information 300 is associated with such products. The change control information 300, the lot information 301, and the lot trace information 302 are stored in the lot information task database 253 in association with each other as above.

The medicine management apparatus 100 further includes a transmission unit 254 that integrates pieces of the information associated as described above and transmits the integrated information to the output apparatus 108. A transmission information accumulation database 255 may be provided to store and accumulate the transmitted information. Although which information is accumulated among pieces of the transmitted information is not particularly limited, an item code, a lot number, and change control number information can be exemplified as applied change control number information 304.

The output apparatus 108 may be any apparatus capable of transmitting the above information to a user, and a display device such as a monitor, a printing device such as a printer, an E-mail automatic transmission device, a FAX automatic transmission device, or the like can be exemplified.

==Medicine Management Method==

A medicine management method according to an embodiment of the invention is a medicine management method using a medicine management apparatus connected so as to be capable of receiving information from a production management apparatus that manages lot information including item information and a lot number of a medicine and lot trace information, a change management apparatus that manages change control information including a change control number of the medicine, an importing country, and product information, and an input apparatus that inputs the lot number of the medicine and the change control number, and includes: receiving the lot information from the production management apparatus; receiving the lot trace information from the production management apparatus; receiving the change control information from the change management apparatus; and associating the lot information including the lot number and the lot trace information with the change control information including the change control number according to the lot number and the change control number to be applied to a product having the lot number input from the input apparatus. The method will be described in detail hereinafter.

First, the medicine management apparatus receives the lot information from the production management apparatus, the lot trace information from the production management apparatus, and the change control information from the change management apparatus. Although the order of reception of these pieces of information is not limited, it is preferable that the medicine management apparatus automatically receive each piece of information from each apparatus at a predetermined interval. The predetermined interval is not particularly limited, and may be selected by a user as appropriate. For example, information may be updated at intervals of once every five minutes to 24 hours such as once every 24 hours, once every 12 hours, once every 6 hours, once every 3 hours, once every hour, once every 30 minutes, once every 15 minutes, and once every five minutes. This update allows the user to always use the latest information. When updating information, information may be overwritten regarding products with the same lot number as the same product. Alternatively, information may include data such as a production lot registration date and a production order confirmation date, and the entire information may be stored. These pieces of data can be used to determine whether the information is new.

The medicine management apparatus stores the received lot information, lot trace information, and change control information in first to third storage devices. There is no particular limitation on a method of processing and storing information when storing the information. For example, each information may be stored in each storage device as a database.

Next, the lot information including the lot number and the lot trace information are associated with the change control information including the change control number according to the lot number and the change control number to be applied to the product having the lot number, which have been input from the input apparatus. If there is a plurality of pieces of information to be associated, the new and old thereof may be determined using the production lot registration date, the production order confirmation date, or the like as described above. Further, the lot number and the change control number input from the input apparatus may be temporarily stored in a fourth storage device. Further, the user who inputs the information from the input apparatus may simultaneously input and instruct a desired association method. For example, examples of instructions can include a timing at which an association is to be applied, lot information to be exempted from an association, to make an association only with specific lot information, and to make an association continuously from now on.

After the lot information and the lot trace information are associated with the change control information, the change control information may be associated with the lot information of the product including the same item information as the above lot information and lot trace information. Alternatively, the change control information may be associated with lot information of a product before or after movement regarding movement information included in the lot trace information. In this case, if the change control information is updated so as to cancel the movement, the association between the lot information and the change control information may be canceled accordingly. Alternatively, the change control information may be associated with lot information of a raw material and/or a processed product included in the lot trace information. As a result, the change control information is associated with related products such as the product before or after movement, the product having the same item, the product such as the material, the raw material, and the processed product in a manufacturing process.

The lot information, lot trace information, and change control information associated in this manner are integrated and output from the output apparatus. Therefore, the user can easily refer to the change control number to be applied to each production lot in real time. For example, when exporting a medicine, it is possible to simultaneously know an application status in an importing country for the medicine manufactured by a specific manufacturing method, and to immediately determine whether the medicine is exportable. Based on such visualized information, the user can determine whether to export or not, and it is possible to reduce the accident of exporting a medicine manufactured by a manufacturing method unapproved by a certain country to the country.

1. A medicine management apparatus connected so as to be capable of receiving information from:
a production management apparatus that manages lot information including a lot number of a medicine and lot trace information;
a change management apparatus that manages change control information including a change control number of the medicine, an importing country, and product information; and
an input apparatus that inputs the lot number of the medicine and the change control number,
the medicine management apparatus comprising:
a first storage unit that stores the lot information received from the production management apparatus;
a second storage unit that stores the lot trace information received from the production management apparatus;
a third storage unit that stores the change control information received from the change management apparatus; and
a data management unit that manages the lot information including the lot number and the lot trace information in association with the change control information including the change control number according to the lot number and the change control number to be applied to a product having the lot number input from the input apparatus.

2. The medicine management apparatus according to claim 1, further comprising a fourth storage unit that stores the lot number and the change control number to be applied to the product having the lot number input from the input apparatus.

3. The medicine management apparatus according to claim 1, wherein first lot information out of the lot information stored in the first storage unit is updated by second lot information out of the lot information received from the production management apparatus.

4. The medicine management apparatus according to claim 3, wherein the data management unit manages the second lot information in association with the change control information associated with the first lot information.

5. The medicine management apparatus according to claim 1, wherein first lot trace information out of the lot trace information stored in the second storage unit is updated by second lot trace information out of the lot trace information received from the production management apparatus.

6. The medicine management apparatus according to claim 5, wherein the data management unit manages the second lot trace information in association with the change control information associated with the first lot trace information.

7. The medicine management apparatus according to claim 1, wherein first change control information out of the change control information stored in the third storage unit is updated by second change control information out of the change control information received from the change management apparatus.

8. The medicine management apparatus according to claim 7, wherein the data management unit manages the second change control information in association with the lot information and the lot trace information associated with the first change control information.

9. The medicine management apparatus according to claim 1, wherein the data management unit manages the change control information including the change control number to be applied to a predetermined product in association with the lot information of a raw material of the product and/or a processed product.

10. The medicine management apparatus according to claim 1, wherein
the lot information includes item information, and
the data management unit manages the change control information including the change control number to be applied to a predetermined product in association with the lot information of a product including the same item information as the product.

11. The medicine management apparatus according to claim 1, wherein
the lot trace information includes movement information, and
the data management unit manages the change control information including the change control number to be applied to a predetermined product in association with the lot information of the product before or after movement.

12. The medicine management apparatus according to claim 11, wherein the association of the change control information with the lot information of the product before or after the movement is released when the movement information is deleted.

13. The medicine management apparatus according to claim 1, wherein an output apparatus is connected to the medicine management apparatus so as to be capable of receiving information.

14. A non-transitory computer readable storage medium storing a program causing a computer to perform a method comprising the steps of:
receiving information from:
a production management apparatus that manages lot information including item information and a lot number of a medicine and lot trace information;
a change management apparatus that manages change control information including a change control number of the medicine, an importing country, and product information; and
an input apparatus that inputs the lot number of the medicine and the change control number,
and the program causing the computer to execute:
receiving the lot information from the production management apparatus;
receiving the lot trace information from the production management apparatus;
receiving the change control information from the change management apparatus; and
associating the lot information including the lot number and the lot trace information with the change control information including the change control number according to the lot number and the change control number to be applied to a product having the lot number input from the input apparatus.

15. (canceled)
